(19) United States
(12) Patent Application Publication
(10) Pub. No.: US 2022/0029086 A1
(43) Pub. Date: Jan. 27, 2022

(54) PRESENCE DETERMINATION DEVICE

(71) Applicant: Sumitomo Riko Company Limited, Aichi (JP)

(72) Inventor: Atsuki SHIMIZU, Aichi (JP)

(73) Assignee: Sumitomo Riko Company Limited, Aichi (JP)

(21) Appl. No.: 17/496,738

(22) Filed: Oct. 7, 2021

Related U.S. Application Data
(63) Continuation of application No. PCT/JP2020/029522, filed on Jul. 31, 2020.

(30) Foreign Application Priority Data
Aug. 30, 2019 (JP) 2019-158494

(51) Int. Cl. H01L 41/113; A61B 5/11; A61B 5/00

(52) U.S. Cl. CPC H01L 41/1132; A61B 5/6892; A61B 5/1102

(57) ABSTRACT

There is provided a presence determination device. The presence determination device determines whether or not a living body is present on a detection region, the presence determination device including: a flexible piezoelectric sensor sheet that is laid out in the detection region and outputs a detection signal corresponding to input vibration; a ballistocardiographic signal acquisition unit that extracts a ballistocardiographic signal corresponding to ballistocardioaction from the detection signal of the piezoelectric sensor sheet ; and an absence determination unit that executes an absence determination indicating that the living body is not present on the detection region, based on the ballistocardiographic signal. When a state where the ballistocardiographic signal is lower than an absence threshold value α lasts beyond an absence determination time, the absence determination unit executes the absence determination.

### CROSS-REFERENCE TO RELATED APPLICATION

The present application is a continuation of PCT/JP2020/029522, filed on Jul. 31, 2020, and is related to and claims priority from Japanese patent application no. 2019-158494, filed on Aug. 30, 2019. The entire contents of the aforementioned application are hereby incorporated by reference herein.

### BACKGROUND

### Technical Field

The present disclosure relates to a presence determination device that determines whether or not a living body is present on a detection region.

### Related Art

For example, in checking whether or not a patient (person requiring caregiving) is on a bed in a medical or caregiving field, further, checking a vacant seat in a place such as a restaurant, a theater, or a subway in which there are multiple seats, there is a demand for determination of whether or not a person is present in a specific region without an effort such as confirmation or the like by human in some cases.

In addition, for example, sleep quality is effectively determined by measuring how many times or how much time a person gets up at night and leaves a bed.

In this respect, there is proposed a presence determination device that determines whether or not a person is present on a detection region such as a bed or a seat of a chair, based on a detection result of pressure obtained by using a pressure-sensitive sensor. For example, in Japanese Patent Laid-Open No. 2015-8920 (Patent Literature 1), a pressure sensor is arranged in bedding, and whether or not a user is present on the bedding is determined based on a body load (body pressure) of the user which is detected by the pressure sensor.

However, regarding detection performed by a pressure-sensitive sensor, when a user such as a patient is light in weight, there is concern that detection precision will decrease.

In addition, when a cushion such as a bed pad or a sitting cushion is interposed between a pressure-sensitive sensor and a user, detection precision of pressure also decreases.

Furthermore, when pressure applied to a pressure-sensitive sensor is temporarily reduced or released during a body movement such as turning-over or re-sitting, an erroneous determination indicating that a user leaves a detection region can be executed, or an erroneous determination indicating that the user makes the body movement such as turning-over or re-sitting can be executed when the user leaves the detection region.

### SUMMARY

The present disclosure is to provide a presence determination device that is capable of determining presence and absence of a user on a detection region with high precision.

Hereinafter, aspects provided for grasping the present disclosure will be described; however, the aspects to be described below are provided as examples such that a reasonable combination thereof can be employed, a plurality of configurational elements described in the aspects can also be recognized and employed independently as much as possible, and a reasonable combination between any configurational elements described in different aspects can also be employed. Hence, in the present disclosure, various different aspects can be realized without being limited to the aspects described below.

### BRIEF DESCRIPTION OF THE DRAWINGS

FIG. 1 is a diagram for illustrating a presence determination device as a first embodiment of the present disclosure.

FIG. 2 is a view for illustrating a piezoelectric sensor sheet which configures the presence determination device illustrated in FIG. 1.

FIG. 3 is a plan view illustrating a sensor main body which configures the piezoelectric sensor sheet illustrated in FIG. 2.

FIG. 4 is a cross-sectional view taken along line IV-IV in FIG. 3.

FIG. 5 is a graph illustrating a specific example of a vibration waveform including ballistocardioaction acquired by the piezoelectric sensor sheet.

FIG. 6 is a graph illustrating a waveform of a ballistocardiographic component extracted from the vibration waveform illustrated in FIG. 5.

FIG. 7 is a graph illustrating a signal amplified by a signal amplification unit, the signal being obtained by the waveform illustrated in FIG. 6.

FIG. 8 is a graph illustrating a ballistocardiographic signal which is used to describe presence or absence in states of (i) to (v).

FIG. 9 is a graph illustrating a waveform of a heartbeat component extracted from the amplified signal illustrated in FIG. 7.

FIG. 10 is a diagram for illustrating a presence determination device as a second embodiment of the present disclosure.

FIG. 11 is a graph illustrating a specific example of a vibration waveform including ballistocardioaction acquired by a piezoelectric sensor sheet.

FIG. 12 is a flowchart illustrating a presence-absence determination in the presence determination device illustrated in FIG. 10.

FIG. 13 is a graph illustrating an enlarged time zone T1 when bed-leaving is determined in the vibration waveform illustrated in FIG. 11.

FIG. 14 is a graph of a power spectrum calculated by performing frequency analysis on the vibration waveform illustrated in FIG. 13.

FIG. 15 is a graph illustrating an enlarged time zone T2 when bed-staying is determined in a state where respiration is stopped in the vibration waveform illustrated in FIG. 11.

FIG. 16 is a graph of a power spectrum calculated by performing frequency analysis on the vibration waveform illustrated in FIG. 15.

FIG. 17 is a graph illustrating an enlarged time zone T3 when bed-staying is determined in a state where respiration is found in the vibration waveform illustrated in FIG. 11.

FIG. 18 is a graph of a power spectrum calculated by performing frequency analysis on the vibration waveform illustrated in FIG. 17.

### DESCRIPTION OF THE EMBODIMENTS

Hereinafter, in order to more specifically clarify the present disclosure, embodiments of the present disclosure will be described in detail with reference to the drawings.

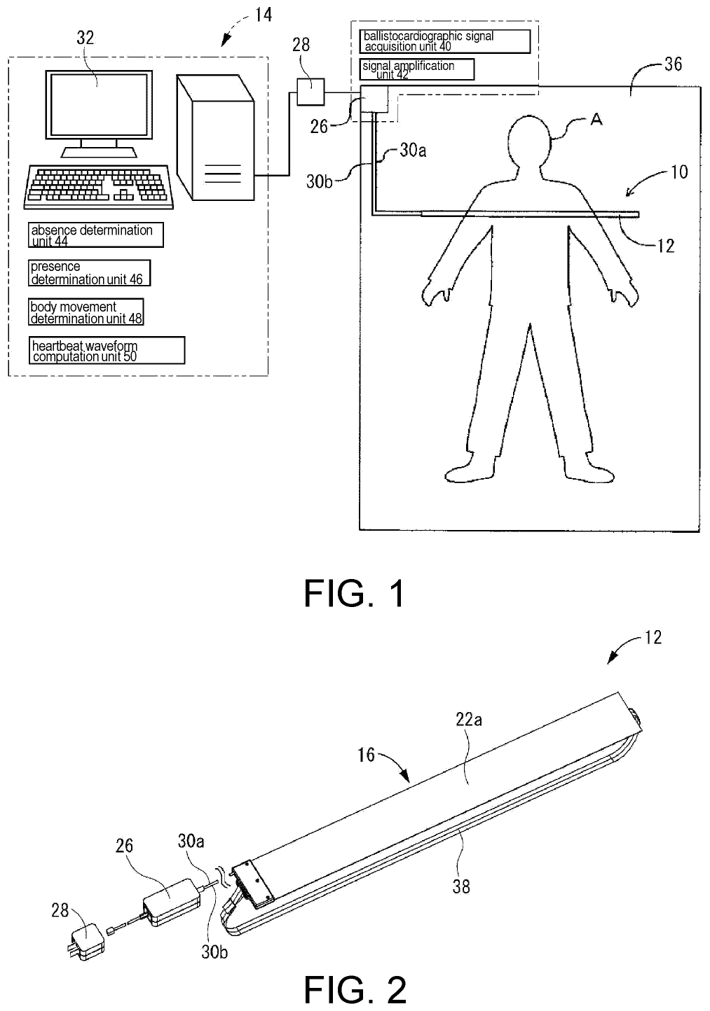

First, FIG. 1 illustrates a presence determination device 10 as a first embodiment of the present disclosure. The presence determination device 10 is configured to include a piezoelectric sensor sheet 12 to which a small body movement of a body due to respiration, a heartbeat, or the like or a relatively large body movement such as turning over of a user A serving as a living body is input and which outputs a detection signal corresponding to the input body movement (vibration), and an analyzer 14 that analyzes the detection signal output from the piezoelectric sensor sheet 12.

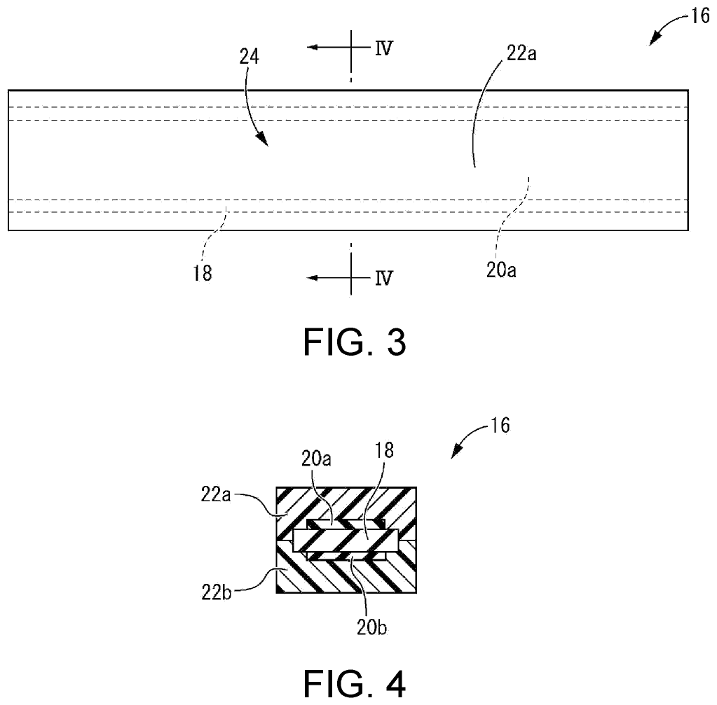

More specifically, the piezoelectric sensor sheet 12 has a structure as illustrated in FIG. 2, for example, and includes a flexible sensor main body 16 having a substantially rectangular sheet shape. As illustrated in FIGS. 3 and 4, the sensor main body 16 includes a piezoelectric layer 18, a pair of electrode layers 20a and 20b, and a pair of protective layers 22a and 22b.

The piezoelectric layer 18 can be made of a material such as ceramic, a synthetic resin, a rubber elastic body (including an elastomer) and is configured by a rubber elastic body in the embodiment. The rubber elastic body employed as the material of the piezoelectric layer 18 preferably contains at least one selected from crosslinked rubbers and thermoplastic elastomers, for example. Examples thereof include urethane rubber, silicone rubber, nitrile rubber (NBR), hydrogenated nitrile rubber (H-NBR), acrylic rubber, natural rubber, isoprene rubber, ethylene-propylene-diene rubber (EPDM), ethylene-vinyl acetate copolymer, ethylene-vinyl acetate-acrylic ester copolymer, butyl rubber, styrene-butadiene rubber, fluoro-rubber, epichlorohydrin rubber, or the like. In addition, an elastomer modified by introducing a functional group or the like may be used. The modified elastomer is preferably, for example, a hydrogenated nitrile rubber having at least one selected from the group consisting of a carboxyl group, a hydroxyl group, and an amino group.

In addition, the piezoelectric layer 18 contains piezoelectric particles. The piezoelectric particles are particles of a compound having piezoelectricity. A ferroelectric substance having a perovskite crystal structure is known as the compound having piezoelectricity, and the compound can be preferably, for example, one or more types of mixtures of barium titanate, strontium titanate, potassium niobate, sodium niobate, lithium niobate, potassium sodium niobate, potassium sodium lithium niobate, lead zirconate titanate (PZT), barium strontium titanate (BST), bismuth lanthanum titanate (BLT), and strontium bismuth tantalate (SBT).

The electrode layers 20a and 20b preferably have flexibility to be deformed in compliance with the piezoelectric layer 18. The piezoelectric layers 20a and 20b can be made of, for example, a conductive material obtained by compounding a conductive substance with a binder, a conductive fiber, or the like. As the binder, the same material as the crosslinked rubber and the thermoplastic elastomer which configure the piezoelectric layer 18 described above can be employed.

In addition, the conductive substance compounded in the electrode layers 20a and 20b is not limited thereto and can be appropriately selected from, for example, metal particles made of gold, copper, nickel, rhodium, palladium, chromium, titanium, platinum, iron, an alloy thereof, and the like, metal oxide particles made of zinc oxide, titanium oxide, or the like, metallic carbide particles made of titanium carbonate, metallic nanowires made of silver, gold, copper, platinum, nickel, or the like, or a conductive carbon material such as carbon black, carbon nanotubes, graphite, thin-layer graphite, or graphene.

As a material of the protective layers 22a and 22b, the same material as the crosslinked rubber and the thermoplastic elastomer which configure the piezoelectric layer 18 described above can be employed.

In the embodiment, all of the piezoelectric layer 18, the electrode layers 20a and 20b, and the protective layers 22a and 22b have a thin rectangular plate shape. The electrode layers 20a and 20b are attached to both sides of the piezoelectric layer 18 in a thickness direction thereof, and the protective layers 22a and 22b are attached to both sides of the piezoelectric layer 18 and the protective layers 20a and 20b in the thickness direction thereof In this manner, the piezoelectric layer 18 and the electrode layers 20a and 20b are embedded inside the protective layers 22a and 22b without being exposed outside. The sensor main body 16 has such a structure described above and is formed into a substantially rectangular sheet shape having a thin thickness.

Besides, a region where the piezoelectric layer 18 and the electrode layers 20a and 20b overlap each other in the thickness direction at a central part of the sensor main body 16 in a width direction thereof forms a pressure sensitive unit 24. A load is applied to the pressure sensing unit 24, and thereby an electric charge is generated.

In addition, the piezoelectric sensor sheet 12 of the embodiment includes a controller 26 and a connector 28. The electrode layers 20a and 20b and the controller 26 are electrically connected to each other by wirings 30a and 30b, and the controller 26 is electrically connected to the analyzer 14 via the connector 28.

The controller 26 includes, for example, an amplification unit, an A/D converter, or the like. The amplification unit amplifies an output (voltage) of the piezoelectric sensor sheet 12. The A/D converter converts an output amplified by the amplification unit from an analog signal to a digital signal. However, the output of the piezoelectric sensor sheet 12 may be converted from the analog signal to the digital signal by the A/D converter and then may be amplified by the amplification unit.

The analyzer 14 needs to be capable of computing a signal (that is, digital signal converted by the A/D converter) controlled by the controller 26 and can be realized by a computer including a monitor unit 32 and a computer program which is installed in the corresponding computer to execute an operation, for example.

Here, in the embodiment, the piezoelectric sensor sheet 12 is laid out to extend in a width direction of a bed 36 as a detection region on which the user A lies down. In particular, in the embodiment, the piezoelectric sensor sheet 12 includes a belt portion 38 having an adjustable length, and the belt portion 38 is wound around a mattress of the bed 36 such that the piezoelectric sensor sheet 12 is fixed to the top of the mattress, and a sheet covers the top of the mattress to which the piezoelectric sensor sheet 12 is fixed. In this manner, the user A lies down on the piezoelectric sensor sheet 12 without coming into direct contact with the piezoelectric sensor sheet 12, and the piezoelectric sensor sheet 12 is provided in the vicinity of the chest of the user A in the embodiment. In this manner, even when a posture of the user A is changed in a certain degree due to turning over or the like, for example, ballistocardioaction of the user A is easy to acquire.

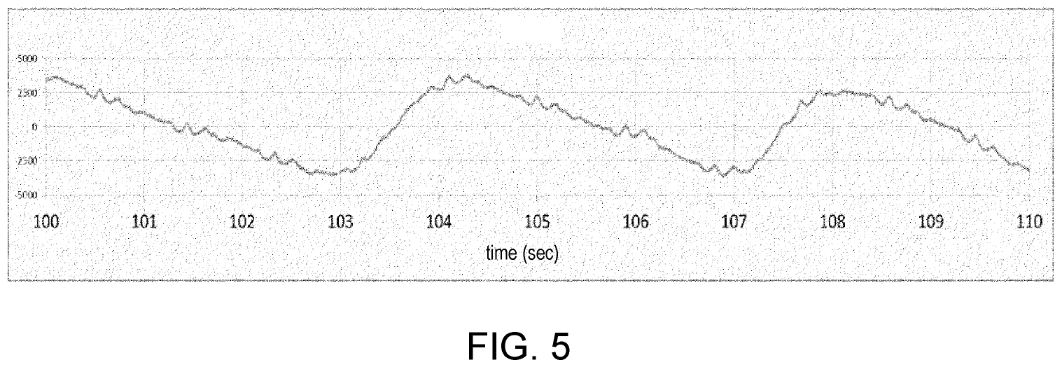

Besides, when a body movement (vibration) in a restricted or general meaning is input to the pressure sensing unit 24 of the piezoelectric sensor sheet 12 due to respiration, a heartbeat, turning-over, or the like of the user A, an electric charge is generated at the piezoelectric layer 18, and the generated electric charge is to be detected as a change in voltage or current by the controller 26. A specific example of a relationship between time and the electric charge (signal) detected by the controller 26 is illustrated as a graph in FIG. 5. That is, a detection signal illustrated in the graph of FIG. 5 is derived from the body movement in the general meaning, such as the respiration, the heartbeat, the turning-over, or the like. Incidentally, the detection signal illustrated in FIG. 5 is raw data which is not processed or corrected.

Hereinafter, a specific example of a procedure for determining whether or not the user A is present on the bed 36 based on ballistocardioaction of the user A will be described by using the presence determination device 10.

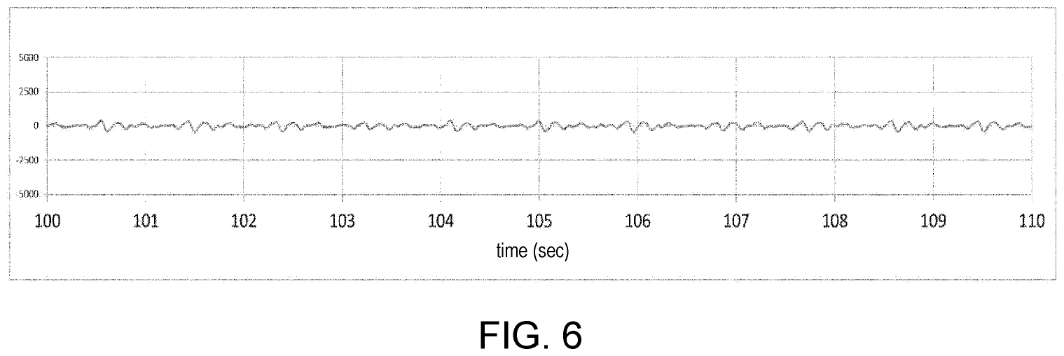

First, the electric charge (signal) generated at the piezoelectric layer 18 is detected via a high-pass filter (ballistocardiographic filter) which cuts off a frequency component lower than 4 Hz (passes a frequency component equal to or higher than 4 Hz). That is, in general, the heartbeat is vibration of about 1 Hz; however, the ballistocardioaction (vibration of a body due to the heartbeat) is vibration equal to or higher than 4 Hz. Hence, a component of the body movement (ballistocardioaction) due to the heartbeat of body movements in the general meaning, such as the respiration, the heartbeat, the turning-over, or the like is extracted, by using the high-pass filter that passes a frequency component equal to or higher than 4 Hz or a bandpass filter that passes a frequency component in a frequency range of 4 Hz to 20 Hz. Incidentally, FIG. 6 illustrates a waveform obtained at a time point when only a component derived from the ballistocardioaction is extracted after passing through the high-pass filter (or bandpass filter).

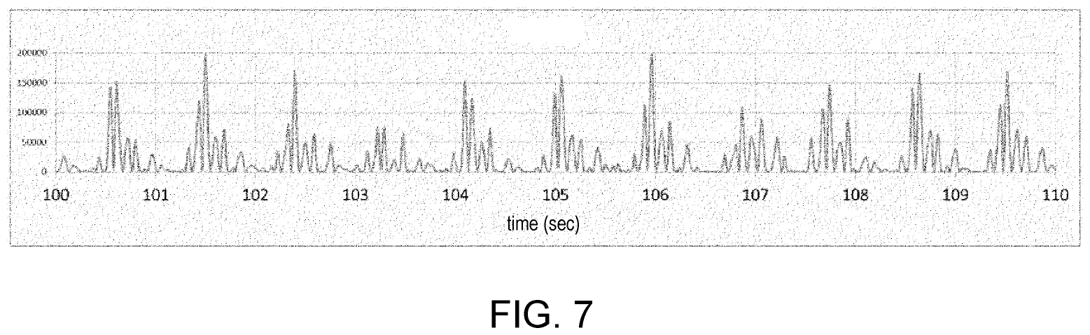

Next, square computation is performed by squaring and amplifying a signal value obtained through the high-pass filter that passes a frequency component equal to or higher than 4 Hz (or bandpass filter that passes a frequency component in a frequency range of 4 Hz to 20 Hz). In this manner, a value on the vertical axis of the graph in FIG. 6 is squared to emphasize a peak of the vibration of the ballistocardioaction. FIG. 7 illustrates a waveform obtained by emphasizing the corresponding ballistocardioaction.

Incidentally, the high-pass filter or the bandpass filter are provided in the controller 26 of the piezoelectric sensor sheet 12 such that a ballistocardiographic signal at a desired frequency is acquired, for example. Hence, a ballistocardiographic signal acquisition unit 40 that extracts a ballistocardiographic signal corresponding to the ballistocardioaction from the detection signal of the piezoelectric sensor sheet 12 may be provided in the controller 26. Subsequently, the ballistocardiographic signal converted into a digital signal by the A/D converter may be transmitted to the computer of the analyzer 14, or the following computation may be performed in the computer. Incidentally, a signal amplification unit 42 that amplifies the ballistocardiographic signal may be provided in the controller 26 or may be configured to include the computer of the analyzer 14 and a program which is installed in the computer.

Here, a representative value within a predetermined time is calculated with respect to the squared and emphasized ballistocardiographic signal (FIG. 7). As the representative value, any one of a maximum value, a minimum value, a mean value, a median value, and a sum thereof within the predetermined time is employed. In the embodiment, a mean value in the previous 0.1 seconds is calculated. In the following description, the representative value of the ballistocardiographic signal is described simply as the ballistocardiographic signal.

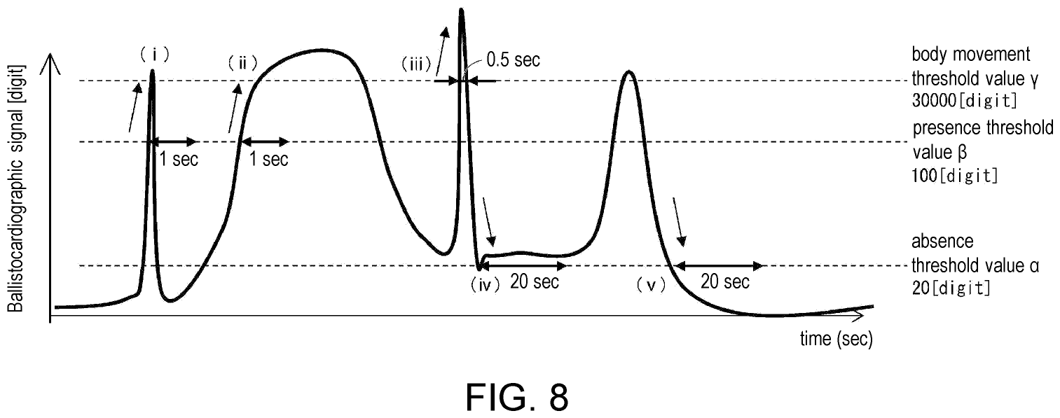

FIG. 8 illustrates an example of the representative value calculated from the ballistocardiographic signal by measuring a ballistocardiographic signal of the user A for the predetermined time. Incidentally, FIG. 8 illustrates a graph for describing states of (i) to (v) to be simply described below, and a value on the vertical axis or the horizontal axis of the graph is not precise value. In addition, in the embodiment, an absence threshold value α, a presence threshold value β, or a body movement threshold value γ which is compared to the ballistocardiographic signal is set in advance. Besides, a state of the user A on the bed 36 is to be determined depending on a magnitude relationship between the ballistocardiographic signal, the absence threshold value α, the presence threshold value β, and the body movement threshold value γ and the duration of the magnitude relationship.

That is, when a state where the ballistocardiographic signal is lower than the absence threshold value α lasts beyond an absence determination time, a determination indicating that the user A is not present (absent) on the bed 36 is to be executed. In the embodiment, the absence threshold value α is set to 20 [digit]. Incidentally, digit represents a minimum display unit of a digital measurement instrument.

In addition, the absence determination time is preferably set to 0.5 seconds or longer. When the absence determination time is shorter than 0.5 seconds, there is concern that an erroneous determination of absence will be executed when a change in posture or false detection results in temporary lowering of a level of the ballistocardiographic signal. Further, the absence determination time is preferably set to 60 seconds or shorter. When the absence determination time is longer than 60 seconds, there is concern that temporary leaving from the bed 36 will not be determined as the absence, for example. The absence determination time is set, more preferably, in a range of 5 seconds to 45 seconds and is set to 20 seconds in the embodiment.

Furthermore, when a state where the ballistocardiographic signal is higher than the presence threshold value β lasts beyond a presence determination time, a determination indicating that the user A is present on the bed 36 is to be executed. As the presence threshold value β, a value equal to the absence threshold value α may be employed; however, it is preferable that the presence threshold value β and the absence threshold value α be independently set to different values from each other. In addition, the presence threshold value β is preferably equal to or larger than three times the absence threshold value α, and the presence threshold value β is set to 100 [digit] in the embodiment.

Further, the presence determination time is preferably set to 0.5 seconds or longer. When the presence determination time is shorter than 0.5 seconds, there is concern that an erroneous determination of presence will be executed when vibration from the outside is input.

In addition, the presence determination time is preferably set to 30 seconds or longer. When the presence determination time is longer than 30 seconds, there is concern that temporary staying on the bed 36 is not determined as the presence, for example. The presence determination time is more preferably set in a range of 0.5 seconds to 15 seconds and is set to 1 second in the embodiment.

In addition, when the ballistocardiographic signal is higher than the body movement threshold value γ, and presence of the user A on the bed 36 for a predetermined time (for example, 30 seconds) before and after the ballistocardiographic signal is higher than the body movement threshold value γ is determined, a determination indicating that the user A makes body movement in the restricted meaning, such as turning-over on the bed 36, is to be executed. Alternatively, a body movement determination time may be set, and a determination indicating that the user A makes a body movement (turning over or re-sitting) on the bed 36 may be executed, when a state where the ballistocardiographic signal is higher than the body movement threshold value γ lasts beyond the body movement determination time. In the embodiment, the body movement determination time is set to 0.5 seconds.

Incidentally, the body movement threshold value γ is preferably set to a value larger than the presence threshold value β, and the body movement threshold value γ is set to 30,000 [digit] in the embodiment.

FIG. 8 illustrates states of (i) to (v) corresponding to the magnitude relationship between the ballistocardiographic signal, the absence threshold value α, the presence threshold value β, and the body movement threshold value γ and the duration of the magnitude relationship. Hereinafter, the states of (i) to (v) will be described.

In (i) of FIG. 8, the ballistocardiographic signal is smaller than the absence threshold value α in the previous state, and thus a determination indicating that the user A is not present (absent) on the bed 36 is executed. Subsequently, since the ballistocardiographic signal exceeds the presence threshold value β, but the state does not last beyond the presence determination time (1 second), a determination indicating that the user A is not present on the bed 36 is executed in (i). For example, such a temporary peak can occur due to a touch or the like of the user A on bedding on the bed 36 or the like.

In (ii) of FIG. 8, since the previous state is determined as absence, the ballistocardiographic signal exceeds the presence threshold value β, and the state lasts beyond the presence determination time (1 second), and a determination indicating that the user A is present on the bed 36 is executed in (ii). Incidentally, in (ii), the ballistocardiographic signal exceeds the body movement threshold value γ, and the state lasts beyond the body movement determination time, but the user A is assumed to be in a state of lying down and starting to sleep on the bed 36 from a relationship with the previous state such that the body movement (turning-over) is not determined.

In (iii) in FIG. 8, the previous state is determined as presence on the bed 36, the ballistocardiographic signal exceeds the body movement threshold value γ, and the state lasts beyond the body movement determination time (0.5 seconds). In addition, in (iii) in FIG. 8, since the ballistocardiographic signal is substantially equal to or slightly lower than the absence threshold value α, but duration thereof is shorter than the absence determination time (20 seconds), the state is not determined as the absence. That is, since the presence of the user A on the bed 36 for the predetermined time (for example, 30 seconds) before and after the ballistocardiographic signal is higher than the body movement threshold value γ is determined, the state of (iii) is determined as the body movement (turning-over or re-sitting).

In (iv) in FIG. 8, as described above, since the ballistocardiographic signal is substantially equal to or slightly lower than the absence threshold value α, but the duration is shorter than the absence determination time (20 seconds), the absence is not determined, and the user A is determined to be present on the bed 36 with consideration for the previous state.

Incidentally, in (iv), a change or the like in posture due to turning-over is considered to primarily lower the ballistocardiographic signal.

In (v) of FIG. 8, since the ballistocardiographic signal is lower than the absence threshold value α, and the state lasts beyond the absence determination time (20 seconds), the absence is determined. That is, consideration of the previous state therewith results in a conclusion of leaving from the bed 36.

Incidentally, in the procedure described above, whether or not the user A is present on the bed 36 is determined from the ballistocardioaction of the user A; however, whether or not the user A is present on the bed 36 may be determined from the heartbeat of the user A.

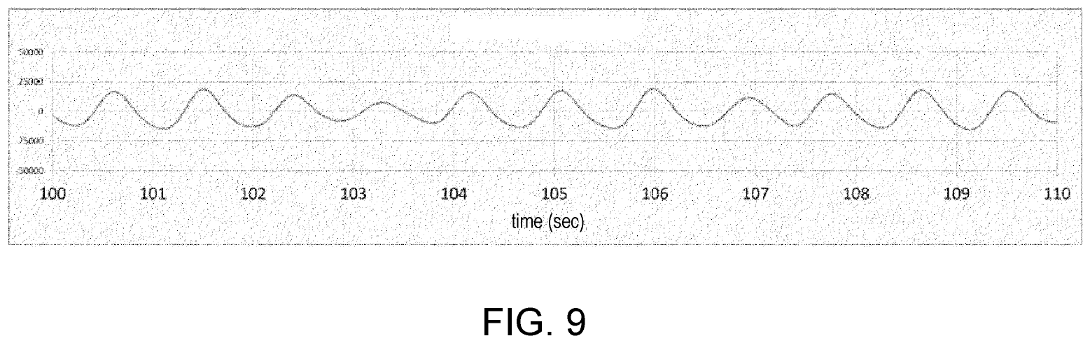

That is, a component derived from the ballistocardioaction is extracted from the detection signal based on an electric charge generated at the piezoelectric layer 18 by the high-pass filter that passes a frequency component equal to or higher than 4 Hz (or bandpass filter that passes a frequency component in a frequency range of 4 Hz to 20 Hz), and a signal value obtained thereby is squared and amplified. Since the component derived from the ballistocardioaction contains a component derived from the heartbeat, the amplified signal value is filtered by the bandpass filter that passes a frequency component in a frequency range of 0.8 Hz to 2.0 Hz, and thereby the component derived from the heartbeat is extracted. In this manner, as illustrated in FIG. 9, a waveform of the heartbeat is obtained.

A representative value within a predetermined time is calculated by using the signal value of the heartbeat which is obtained based on the signal of the ballistocardioaction as described above. The representative value is compared to the absence threshold value, the presence threshold value, the body movement threshold value, or the like set in advance, and thereby whether or not the user A is present on the bed 36 may be determined. Incidentally, as the representative value, a mean value or a maximum value in the previous 10 seconds may be employed. In addition, the absence threshold value, the presence threshold value, and the body movement threshold value can be appropriately set according to a signal value (whether to use the ballistocardiographic signal, to use the heartbeat signal, to employ any one of a maximum value, a minimum value, a mean value, a median value, and a sum, or the like as the representative value) to be compared.

As described above, an absence determination unit 44 that determines that the user A is not present on the bed 36 (executes an absence determination), a presence determination unit 46 that determines that the user A is present on the bed 36 (executes a presence determination), a body movement determination unit 48 that determines whether or not the body movement such as turning-over of the user A is made on the bed 36 (executes a body movement determination), and a heartbeat waveform computation unit 50 that calculates a heartbeat waveform from the amplified ballistocardiographic signal can be configured to include the computer of the analyzer 14 and a program which is installed in the computer, for example.

As described above, since the presence determination device 10 of the embodiment does not determine the presence and absence based on a body load (body pressure) as determined in a pressure sensor in the related art but determines the presence and absence of the user A from the heartbeat or the ballistocardioaction based on the heartbeat and there can be a reduction in concern that the detection precision will be lowered or the erroneous determination will be executed.

In addition, the absence is not determined immediately at a time point when the ballistocardiographic signal is lower than the absence threshold value α, but the absence determination time is set such that the absence is determined when a state where the ballistocardiographic signal is lower than the absence threshold value α lasts beyond the absence determination time. Hence, even when the ballistocardiographic signal is temporarily lowered due to a change in posture such as turning-over, a problem or the like of an erroneous determination of the absence can be avoided.

Further, in the embodiment, the presence determination time is set, and the presence is to be determined when a state where the ballistocardiographic signal is higher than the presence threshold value β lasts beyond the presence determination time. Hence, there can be a reduction in concern that the absence will be determined when the user is present. Furthermore, regarding the body movement such as turning over, the ballistocardiographic signal is higher than the body movement threshold value, and the presence is determined for the predetermined time before and after the state where the ballistocardiographic signal is higher than the body movement threshold value, a determination indicating that the body movement is made is executed. Hence, highly precise determination can be executed, compared to a case where the determination indicating that a body movement is made is executed when the ballistocardiographic signal is simply higher than the body movement threshold value.

In addition, in the embodiment, the ballistocardiographic signal or the heartbeat signal which is compared to the absence threshold value α, the presence threshold value β, or the body movement threshold value γ is set to a representative value within a predetermined time, and a maximum value, a minimum value, a mean value, or median value, or a sum is used as the representative value. The maximum value is employed as the representative value, and thereby a numerical value of the ballistocardiographic signal can be increased overall such that a highly precise determination can be executed. Further, the minimum value is employed as the representative value, and thereby a numerical value of the vibration from the outside which is input as noise can be restricted to a small value such that concern of the erroneous determination can be reduced. Furthermore, the mean value is employed as the representative value, and thereby both an effect obtained when the maximum value is employed and an effect obtained when the minimum value is employed as described above can be achieved such that the presence determination device having a good balance can be provided.

Further, in the embodiment, the absence threshold value α and the presence threshold value β are independently set to different values from each other. That is, the presence threshold value β can be set to a relatively large value, for example. In this manner, a problem of a determination of the presence even in a state of absence due to vibration from the outside which is input as noise can be avoided. Similarly, the absence threshold value α can be set to a relatively small value, for example. In this manner, a problem of a determination of the absence even in a state of presence can be avoided. That is, it is preferable that the absence threshold value α and the presence threshold value β have a large difference therebetween. For example, the difference can be equal to or larger than three times or five times the value or 20 times the value depending on a use state.

Furthermore, amplification of the ballistocardiographic signal by the signal amplification unit 42 can improve the precision. In addition, the heartbeat waveform is calculated using the amplified ballistocardiographic signal, and thereby whether or not the user A is present on the bed 36 can be determined from the heartbeat waveform.

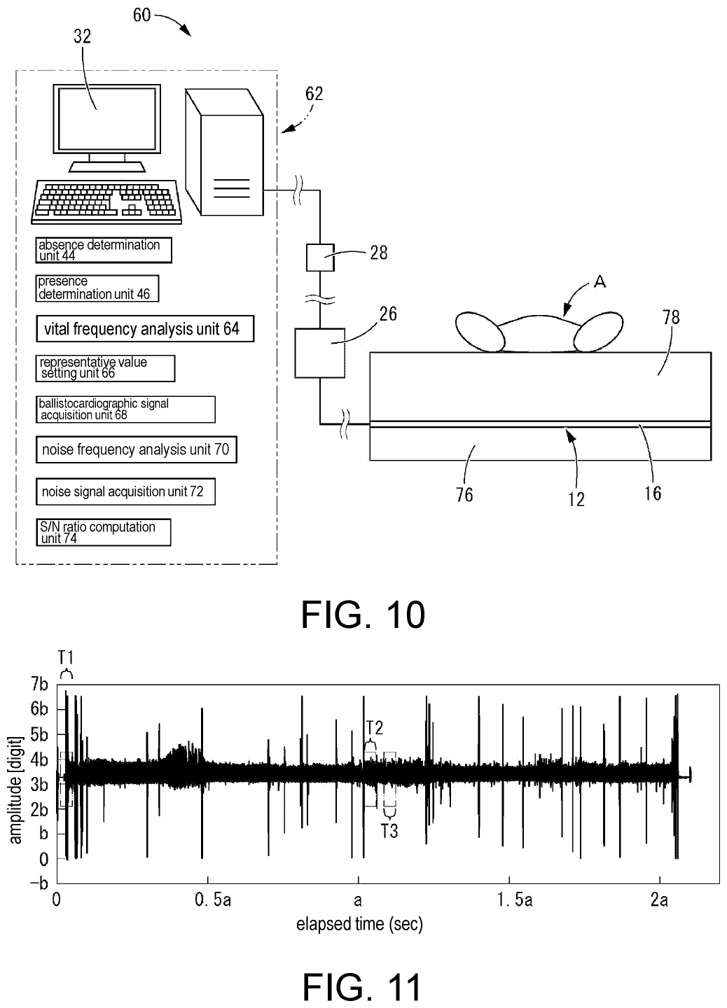

FIG. 10 illustrates a presence determination device 60 as a second embodiment of the present disclosure. In the following description, the same reference signs will be assigned to the substantially same members and parts as those of the first embodiment, and thereby the description thereof will be omitted.

The presence determination device 60 is configured to include a piezoelectric sensor sheet 12 that outputs a detection signal corresponding to a body movement (vibration) of a user A, and an analyzer 62 that analyzes the detection signal output from the piezoelectric sensor sheet 12.

The analyzer 62 is electrically connected to the piezoelectric sensor sheet 12 via a controller 26 with wiring. The analyzer 62 is configured to be capable of computing a signal controlled by the controller 26. The analyzer 62 includes an absence determination unit 44 and a presence determination unit 46.

The analyzer 62 includes a vital frequency analysis unit 64 that performs frequency analysis on the detection signal of the piezoelectric sensor sheet 12 and calculates vital spectra which are power spectra related to ballistocardioaction and respiration of the user A.

The vital frequency analysis unit 64 performs the frequency analysis on the detection signal of the piezoelectric sensor sheet 12, thereby, calculating the vital spectra which are power spectra including both a ballistocardiographic spectrum which is a power spectrum obtained from the ballistocardioaction and a respiration spectrum which is a power spectrum obtained from the respiration. The vital spectra do not need to always include the respiration spectrum. For example, in a case or the like where respiration is temporarily stopped during sleep due to the sleep apnea syndrome while sleeping, the vital spectra does not include the respiration spectrum. The vital frequency analysis unit 64 executes a frequency analyzing process by the fast Fourier transform (FFT) and is configured of a computer in which a frequency analysis program or the like is installed, for example.

Incidentally, the ballistocardiographic spectrum is mainly found in a frequency range of about 1 Hz to 5 Hz, and the respiration spectrum is mainly found in a frequency range of about 0.1 Hz to 2 Hz. In the frequency analysis, a frequency range for processing a vital spectrum is appropriately set; however, the vital spectrum is calculated as a power spectrum having a frequency range of 0.1 Hz to 5 Hz.

The analyzer 62 includes a representative value setting unit 66 that computes a representative value of the vital spectra calculated by the vital frequency analysis unit 64. The representative value of the vital spectra is not particularly limited and is selected depending on a purpose. For example, a maximum value, a minimum value, a mean value, a median value, a sum, or the like of the vital spectra in a predetermined time is employed as the representative value.

The representative value setting unit 66 of the embodiment executes a process of obtaining a maximum value of the vital spectra as the representative value of the vital spectra. The representative value setting unit 66 is configured of a computer in which a representative-value computation processing program or the like is installed, for example.

The analyzer 62 includes a ballistocardiographic signal acquisition unit 68 that acquires a ballistocardiographic signal based on the representative value of the vital spectra set by the representative value setting unit 66. The ballistocardiographic signal acquisition unit 68 is configured of a computer in which a ballistocardiographic signal generation processing program or the like is installed, for example.

The analyzer 62 includes a noise frequency analysis unit 70 that performs frequency analysis on the detection signal of the piezoelectric sensor sheet 12, thereby, calculating a noise spectrum which is a power spectrum in a frequency range higher than the frequency range (vital frequency range) of the vital spectra. The noise frequency analysis unit 70 executes a frequency analyzing process by the fast Fourier transform (FFT) and is configured of a computer in which a frequency analysis program or the like is installed, for example. Incidentally, the noise spectrum is preferably a power spectrum in a frequency range in which an effect of the ballistocardioaction or the respiration is small and is calculated as a power spectrum in a frequency range of 20 Hz to 25 Hz.

The representative value setting unit 66 computes the representative value of the noise spectrum calculated by the noise frequency analysis unit 70. The representative value of the noise spectrum is not particularly limited and is selected depending on a purpose. For example, a maximum value, a minimum value, a mean value, a median value, a sum, or the like of the noise spectrum in a predetermined time is employed as the representative value. The representative value setting unit 66 of the embodiment executes a process of obtaining a mean value of the noise spectrum as the representative value of the noise spectrum.

The analyzer 62 includes a noise signal acquisition unit 72 that acquires a noise signal based on the representative value of the noise spectrum set by the representative value setting unit 66. The noise signal acquisition unit 72 is configured of a computer in which a noise-signal generation processing program or the like is installed, for example.

The analyzer 62 includes an S/N ratio computation unit 74 that calculates a ratio (S/N ratio) of the ballistocardiographic signal as a signal to the noise signal as noise. The S/N ratio computation unit 74 is configured of a computer in which a computation processing program or the like is installed, for example.

Besides, the absence determination unit 44 and the presence determination unit 46 executes a presence-absence determination based on the ratio of the ballistocardiographic signal to the noise signal calculated by the S/N ratio computation unit 74. The absence determination unit 44 compares the calculated S/N ratio to the preset absence threshold value and executes the absence determination when the S/N ratio is lower than the absence threshold value for a predetermined absence determination time or longer. The presence determination unit 46 compares the calculated S/N ratio to the preset presence threshold value and executes the presence determination when the S/N ratio is lower than the presence threshold value for a predetermined presence determination time or longer. Incidentally, the absence threshold value, the presence threshold value, the absence determination time, and the presence determination time are all appropriately set depending on an input size (strength of a signal) of the piezoelectric sensor sheet 12, noise from a surrounding environment, required determination precision, or the like and are not particularly limited.

The presence determination device 60 is used in watching-over or the like of the user A at bedtime and determines whether the user A is present or absent on a bed 36. The bed 36 as a detection region on which the user A lies down has a bed board 76 on which a mattress 78 as a cushion body is mounted, and the sensor main body 16 of the presence determination device 60 is disposed between the bed board 76 and the mattress 78. In short, the sensor main body 16 is disposed on a lower side of the mattress 78, and thus vibration (ballistocardioaction, body movement, or the like) which is applied to the mattress 78 by the user A is indirectly input to the sensor main body 16 via the mattress 78. The mattress 78 may be covered with a sheet for inhibiting wear or dirt from being attached, or both the sensor main body 16 and the mattress 78 can be covered together with the sheet. Incidentally, it is desirable that the sensor main body 16 be disposed in the vicinity of the chest of the user A in the top view, similarly to the first embodiment. The sensor main body 16 may be positioned with respect to the bed board 76 or the mattress 78 by a band, a hook, or the like. In particular, when the sensor main body 16 is applied to a nursing bed having a back raising function, it is desirable that the sensor main body 16 be positioned to inhibit the sensor main body from shifting or dropping out due to back raising. In addition, when the sensor main body 16 is positioned to the bed 36, it is desirable that the sensor main body can be released from positioning, and it is preferable that the sensor main body 16 is detachably positioned to the bed 36.

In a state where the presence determination device 60 is set to the bed 36, a change in input due to a heartbeat, respiration, a body movement, or the like of the user A is detected by the piezoelectric sensor sheet 12. FIG. 11 is a graph illustrating an example of a detection result obtained by the piezoelectric sensor sheet 12. In the graph of FIG. 11, the vertical axis indicates a detected amplitude, that is, strength of the detection signal, and is illustrated by a unit of digit, and the horizontal axis indicates time and is illustrated by a unit of seconds. Incidentally, the graph of FIG. 11 represents a raw waveform of the detection signal output from the piezoelectric sensor sheet 12 and includes a waveform of electromagnetic noise, a waveform of vibration input from a floor to the bed 36, or the like, in addition to vibration waveforms due to the ballistocardioaction, the respiration, the body movement, or the like of the user A.

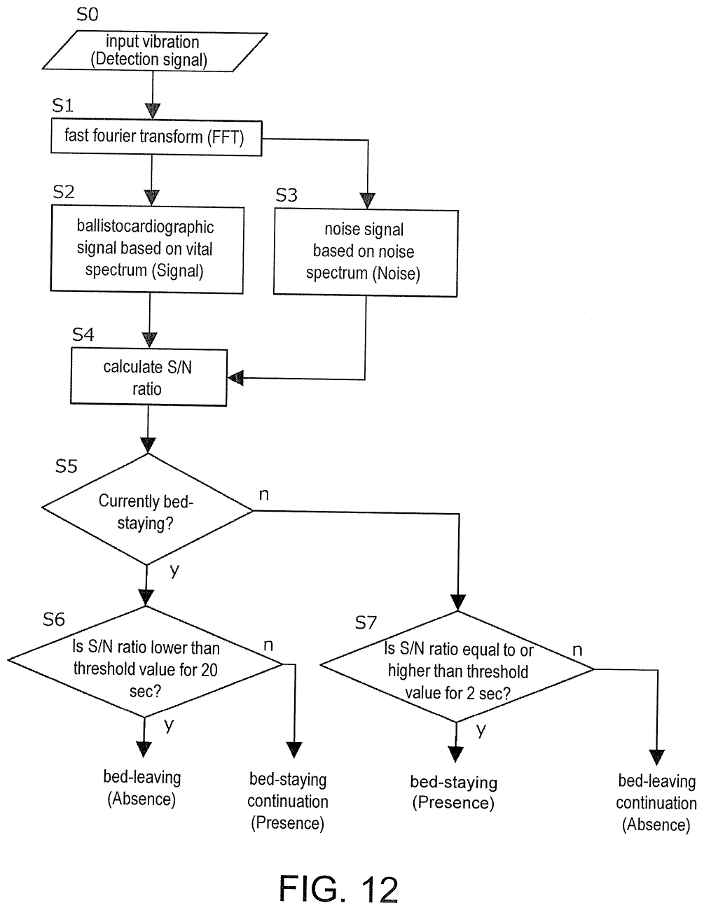

Besides, the analyzer 62 executes a presence-absence determination illustrated in a flowchart of FIG. 12 based on the detection signal transmitted from the piezoelectric sensor sheet 12, thereby, determining whether or not the user A is present on the bed 36. Hereinafter, an example of the presence-absence determination performed by the presence determination device 60 in characteristic three time zones T1, T2, and T3 in FIG. 11 will be described.

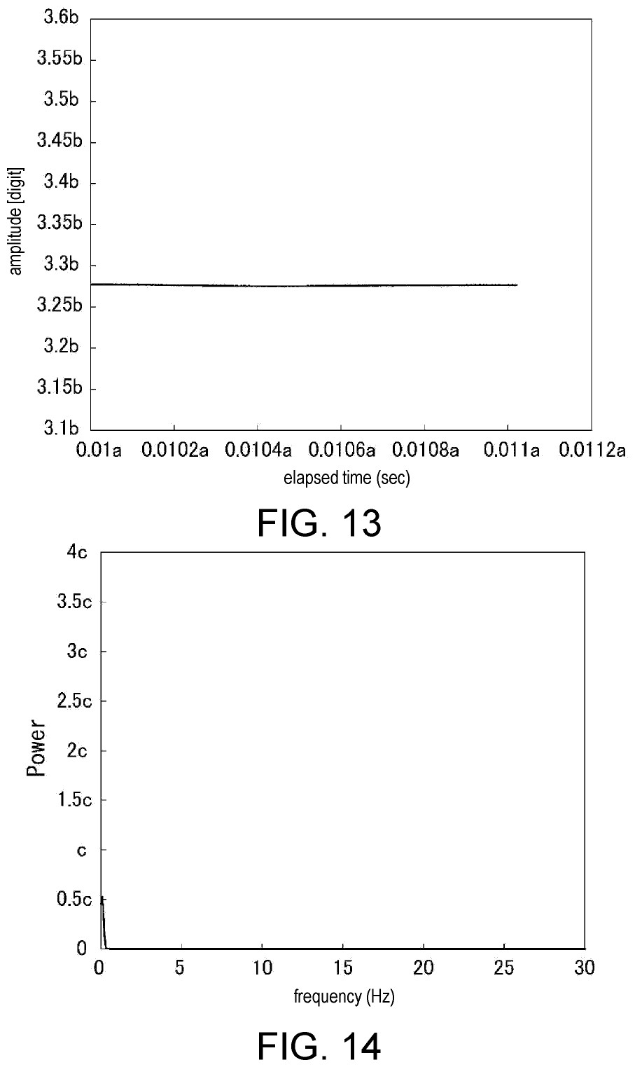

The time zone T1 illustrated in FIG. 11 is a time zone of a bed-leaving state in which the user A is not present on the bed 36. As enlarged in FIG. 13, the waveform of the detection signal output from the piezoelectric sensor sheet 12 is substantially flat in T1.

The detection signal transmitted to the analyzer 62 in Step (hereinafter, S) 0 of FIG. 12 is subjected to a frequency analyzing process using the fast Fourier transform (FFT) in S1 by the vital frequency analysis unit 64 and the noise frequency analysis unit 70. A graph in FIG. 14 illustrates a result from frequency analysis of the detection signal in T1. In the graph of FIG. 14, the horizontal axis indicates a frequency, and the vertical axis indicates strength of a power spectrum. According to the graph of FIG. 14, an increase in power spectrum due to noise at a frequency lower than 0.1 Hz is found; however, the vital spectrum which is the power spectrum in the frequency range of 0.1 Hz to 5 Hz is represented by a very low numerical value. In addition, the noise spectrum which is the power spectrum in the frequency range of 20 Hz to 25 Hz also is represented by a very low numerical value.

In S2, the ballistocardiographic signal acquisition unit 68 acquires the ballistocardiographic signal based on the representative value of the power spectra (vital spectra) in the frequency range of 0.1 Hz to 5 Hz set by the representative value setting unit 66. The representative value of the vital spectra is not particularly limited. For example, a maximum value, a minimum value, a mean value, a median value, or a sum of the vital spectra is employed as the representative value. In the embodiment, the maximum value is employed as the representative value of the vital spectra.

In S3, the noise signal acquisition unit 72 acquires the noise signal based on the representative value of the power spectrum (noise spectrum) in the frequency range of 20 Hz to 25 Hz set by the representative value setting unit 66. The representative value of the noise signal is not particularly limited. For example, a maximum value, a minimum value, a mean value, a median value, or a sum of the noise spectrum is employed as the representative value. In the embodiment, the mean value is employed as the representative value of the noise spectrum. A method for calculating the representative value of the vital spectra and a method for calculating the representative value of the noise spectrum may be the same as each other by employing the mean value in both methods, for example, or may be different from each other as in the embodiment.

In S4, the S/N ratio computation unit 74 of the analyzer 62 calculates, as a signal/noise ratio (S/N ratio), a ratio of the ballistocardiographic signal based on the representative value of the vital spectra to the noise signal based on the representative value of the noise spectrum.

In S5, a determination of whether or not the user A is present on the bed 36 at a time of the previous determination is executed. Incidentally, a determination of presence (y) is obtained at a time of a first determination in S5. That is, in a case in which the presence determination device 60 is used in watching-over or the like of a patient (user A), since bed-staying of the user A immediately after the bedtime is checked by a caregiver or the like, the first determination is executed as the bed-staying. Subsequently, the watching-over of bed-leaving is performed by the presence determination device 60, and thereby the absence determination which is executed for a determination time longer than that of a bed-staying determination and which is unlikely to result in an erroneous determination due to noise or the like enables the first bed-leaving to be precisely detected. Incidentally, when the first determination in S5 indicates the bed-staying (y), a determination result in S6 is a constant presence determination for 20 seconds from a determination start; however, since the time is a short time of 20 seconds, a visual check by the caregiver or the like only for the time is unlikely to bring about a burden, and there is substantially no concern that an erroneous determination for the time results in a serious problem.

However, in the first determination in S5, absence (n) may be determined. In this respect, when an erroneous determination is executed by any chance, a problem is unlikely to arise, since the absence determination executed even though the user A is actually present on the bed 36 means an erroneous determination which is considered to cause a safe check to be performed. In addition, the presence determination time (2 seconds) in S7 is shorter than the absence determination time (20 seconds) set in S6 such that a usual determination can be more rapidly executed, and thus a restraint time of a busy caregiver or the like is more shortened.

In S5, when a determination indicating that the user A is present on the bed 36 (y) is executed, the absence determination unit 44 determines the bed-leaving in S6. That is, when a state where the S/N ratio stored in S4 is smaller than the preset absence threshold value (lower than the absence threshold value) lasts beyond the preset absence determination time, the absence determination is executed, and a determination indicating that the user A leaves the bed 36 (bed-leaving) is executed. On the other hand, when a state where the S/N ratio is smaller than the absence threshold value does not occur or does not last beyond the absence determination time, a determination indicating that the user A is present on the bed 36 (bed-staying continuation) is executed. Incidentally, a degree of the absence threshold value is appropriately set depending on performance of the piezoelectric sensor sheet 12, a level of noise from a surrounding environment, or the like. In addition, a length of the absence determination time is appropriately set depending on demanded difficulty of the occurrence of the erroneous determination and is preferably set to 20 seconds, for example.

In S5, when a determination indicating that the user A is not present on the bed 36 (n) is executed, the presence determination unit 46 determines the bed-staying in S7. That is, when a state where the S/N ratio stored in S4 is larger than the preset presence threshold value (higher than the presence threshold value) lasts beyond the preset presence determination time, the presence determination is executed, and a determination indicating that the user A stays on the bed 36 (bed-staying) is executed. On the other hand, when a state where the S/N ratio is larger than the presence threshold value does not occur or does not last beyond the presence determination time, a determination indicating that the user A is not present on the bed 36 (bed-leaving continuation) is executed. Incidentally, a degree of the presence threshold value is appropriately set depending on performance of the piezoelectric sensor sheet 12, a level of noise from a surrounding environment, or the like. In addition, a length of the presence determination time is appropriately set depending on demanded difficulty of the occurrence of the erroneous determination and is preferably set to 2 seconds, for example. Preferably, the presence determination time is shorter than the absence determination time.

According to the spectrum of T1 illustrated in FIG. 14, the S/N ratio is decreased, and thus the bed-leaving continuation is determined without executing the presence determination. Hence, in T1, a determination indicating that the user A is not present on the bed 36 is executed.

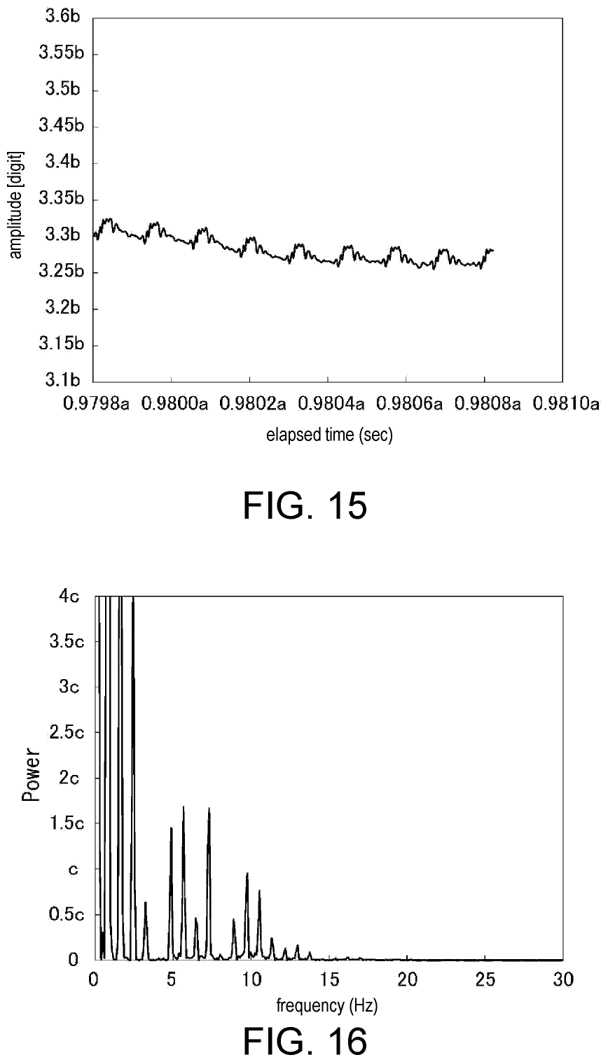

The time zone T2 illustrated in FIG. 11 is a time zone of a bed-staying state in which the user A stays on the bed 36 and is a time zone when respiration of the user A is temporarily stopped. As enlarged in FIG. 15, in T2, a waveform of the detection signal output by the piezoelectric sensor sheet 12 is a regular-shaped waveform of a small amplitude due to the ballistocardioaction derived from the heartbeat without having a waveform of a large amplitude due to the respiration. Incidentally, even in the presence-absence determination of T2, a process of calculation or the like of the representative value or the S/N ratio is executed similar to the presence-absence determination of T1; however, the description of the substantially same process as that of T1 is omitted for simplification of the description.

FIG. 16 illustrates a graph of a result from frequency analysis of the detection signal in T2. In T2, the vital spectrum is a ballistocardiographic spectrum which does not include the respiration spectrum. According to FIG. 16, the vital spectrum is illustrated to about 20 Hz, and particularly a numerical value of the vital spectrum in the frequency range of about 0.1 Hz to 3 Hz is increased. On the other hand, the noise spectrum which is the power spectrum in the frequency range of 20 Hz to 25 Hz also is represented by a very small numerical value similar to T1. Hence, in T2, the S/N ratio which is the ratio of the ballistocardiographic signal to the noise signal is higher than that in T1.

In the flowchart of the determination process illustrated in FIG. 12, regarding the absence determination in T2, the bed-staying is determined in the previous determination in S5. Consequently, in S6, whether or not a state where the S/N ratio is lower than the absence threshold value lasts for 20 seconds or longer is determined. In T2, the S/N ratio is increased due to the ballistocardioaction, and thus the absence determination is denied in the determination of S6, and a determination indicating that the user A is present on the bed 36 is executed.

As described above, even when the respiration of the user is temporarily stopped, and the respiration spectrum is not found, the presence-absence determination can be precisely executed with the ballistocardiographic signal based on the ballistocardiographic spectrum without including the respiration spectrum.

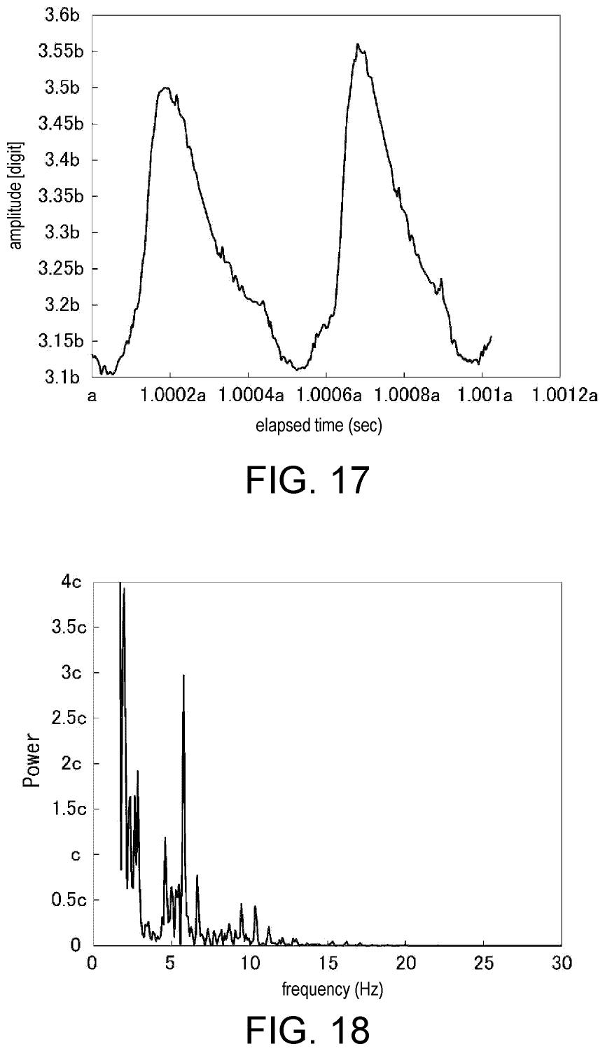

The time zone T3 illustrated in FIG. 11 is a time zone of the bed-staying state in which the user A stays on the bed 36 and is a time zone when the user A breathes. As enlarged in FIG. 17, in T3, a waveform of the detection signal output by the piezoelectric sensor sheet 12 is a waveform obtained by combining a waveform of a large amplitude due to the respiration and a waveform of a small amplitude due to the ballistocardioaction derived from the heartbeat. Incidentally, regarding the presence-absence determination in T3, the description of the same process as that in T1 is also omitted.

FIG. 18 illustrates a graph of a result from frequency analysis of the detection signal in T3. According to FIG. 18, the vital spectra obtained by combining the ballistocardiographic spectrum due to the ballistocardioaction and the respiration spectrum due to the respiration is illustrated to about 20 Hz, and particularly a numerical value of the vital spectra in the frequency range of 0.1 Hz to about 10 Hz is increased. On the other hand, the noise spectrum which is the power spectrum in the frequency range of 20 Hz to 25 Hz is represented by a very low numerical value. Hence, in S3, the S/N ratio which is a ratio of the representative value (maximum value) of the ballistocardiographic signal based on vital spectrum to the representative value (mean value) of the noise signal based on the noise spectrum is more increased than in T1. Further, in T3, since the vital spectra which configure the ballistocardiographic signal have the respiration spectrum in addition to the ballistocardiographic spectrum, the ballistocardiographic signal is more increased than in T2 where the vital spectrum does not include the respiration spectrum, and thus the S/N ratio is more increased.

In the flowchart of the determination process illustrated in FIG. 12, regarding the presence-absence determination in T3, the bed-staying is determined in the previous determination in S5. Consequently, in S6, whether or not a state where the S/N ratio is lower than the absence threshold value lasts for 20 seconds or longer is determined. In T3, the S/N ratio is increased due to the ballistocardioaction and the respiration, and thus the absence determination is denied in the determination of S6, and a determination indicating that the user A is present on the bed 36 is executed.

As described above, when the user A stays on the bed 36 while breathing as usual, a higher S/N ratio can be obtained from the ballistocardiographic signal which is generated based on the vital spectra including the respiration spectrum in addition to the ballistocardiographic spectrum, than the S/N ratio when the respiration is stopped. Consequently, highly precise presence-absence determination which is unlikely to result in the erroneous determination can be executed.

In the presence determination device 60 according to the embodiment, the sensor main body 16 of the piezoelectric sensor sheet 12 is disposed on the lower side of the mattress 78. Hence, even when an input to the sensor main body 16 is cushioned by the mattress 78 to be decreased, a highly precise determination can be realized based on the ballistocardiographic signal by the frequency analysis. Incidentally, in the presence determination device 60 of the embodiment, the sensor main body 16 of the piezoelectric sensor sheet 12 can be disposed on an upper side of the mattress 78.

The sensor main body 16 is laid out on the lower side of the mattress 78, and thereby the sensor main body 16 can be inhibited from being bent in a thickness direction thereof or having a position shifting with respect to the mattress 78, due to a body movement such as bed-entering, bed-leaving, turning-over, or the like of the user A. Moreover, the sensor main body 16 is covered with the mattress 78, and thereby an effect of an external environment such as wind on the sensor main body 16 can be reduced such that the detection signal can be obtained with high precision.

The absence determination unit 44 and the presence determination unit 46 executes the absence determination or the presence determination based on comparison between the threshold value and the S/N ratio which is the ratio of the ballistocardiographic signal to the noise signal. Consequently, when substantially uniform noise (white noise) is input to the entire frequency range, the effect of the corresponding noise is reduced such that precise determination can be executed. However, the absence determination unit 44 and the presence determination unit 46 may execute the absence determination or the presence determination based on comparison between the ballistocardiographic signal and the threshold value. In this case, the noise frequency analysis unit 70, the noise signal acquisition unit 72, and the S/N ratio computation unit 74 can be omitted.

The ballistocardiographic signal acquisition unit 68 acquires the ballistocardiographic signal based on the representative value of the vital spectra, and the noise signal acquisition unit 72 acquires the noise signal based on the representative value of the noise spectrum. In this manner, a highly precise determination of presence and absence, a reduction in erroneous determination due to noise, simplification in computation process of presence-absence determination, or the like is achieved.

In particular, in the embodiment, the representative value of the vital spectrum is set to the maximum value, and the representative value of the noise spectrum is set to the mean value.

In this manner, the high S/N ratio can be obtained, and an effect of noise can be suppressed, when the noise momentarily increases like electromagnetic noise to have the effect.

Incidentally, similarly to the first embodiment, the body movement determination unit may be provided in the analyzer 62 such that the body movement such as the turning-over of the user A may be determined. In this case, the body movement determination unit executes the body movement determination based on a result of comparison between the body movement threshold value and the S/N ratio obtained by the S/N ratio computation unit 74.

As described above, the embodiments of the present disclosure are described; however, the present disclosure is not construed to be limited to the specific description of the embodiments, and the present disclosure can be realized in aspects obtained by performing various modifications, changes, improvements, or the like on the present disclosure based on knowledge of those skilled in the art.

The representative value is a characteristic value briefly indicating a tendency of distribution and includes a calculated representative value such as an arithmetic average value, a geometric average value, a harmonic average value, or a square average value and a positional representative value such as a median value, a mode value, a quartile, a maximum value, or a minimum value. In general, the calculated representative value includes all values to find summarized characteristics, and the positional representative value can represent a characteristic obtained by reducing an effect of an extreme value in data. Hence, when specific noise or the like is considered, for example, a mode value or the like is employed, or the minimum value (maximum noise) is employed, rather than the maximum value, in some cases. In a case of noise or the like (including minimum and maximum noise due to turning over, or the like) which is difficult to identify, the mean value may be employed, or the maximum value, the mean value, the sum, or the like may be employed also with consideration for simplification of computation.

The specific numerical values related to a filtering frequency, the threshold value, the determination time, or the like described in the embodiment are only provided as an example, are not construed to be limited thereto, and can be appropriately adjusted depending on a state, an environment, or the like of a user.

In addition, the amplification of the ballistocardiographic signal by the signal amplification unit is not limited to the square; however, the even-numbered power is preferably used with consideration for a problem of positive and negative. In addition, since the smaller the number, the easier the computation, the square is preferable. However, the signal amplification unit is not an essential unit. For example, whether or not the user A is present on the detection region may be determined based on the signal value of the ballistocardioaction represented by the waveform of FIG. 6 illustrated above.

Further, the detection region is not limited to the bed, and the detection region may be a chair, for example. That is, the presence determination device according to the present disclosure is applied to chairs or seats in a restaurant, a theater, or a train, and thereby vacancy state or the like can be easily checked.

Other Configurations

According to one aspect, there is provided a presence determination device that determines whether or not a living body is present on a detection region, the presence determination device including: a flexible piezoelectric sensor sheet that is laid out in the detection region and outputs a detection signal corresponding to input vibration; a ballistocardiographic signal acquisition unit that extracts a ballistocardiographic signal corresponding to ballistocardioaction from the detection signal of the piezoelectric sensor sheet; and an absence determination unit that executes an absence determination indicating that the living body is not present on the detection region, based on the ballistocardiographic signal. When a state where the ballistocardiographic signal is lower than an absence threshold value lasts beyond an absence determination time, the absence determination unit executes the absence determination.

According to the presence determination device of the aspect, absence is determined based on the ballistocardioaction, and thereby the determination can be executed with higher precision, compared to a determination executed based on pressure due to a body weight or the like of the living body.

In addition, regarding the absence determination indicating that a patient leaves a bed or the like when the leaving of the patient results in a problem in watching-over or the like of the patient, the absence determination time is set, and thus the determination can be executed with higher precision without an immediate determination of absence, even when a change or the like in posture due to simple turning over results in a low amplitude of vibration which is input to the piezoelectric sensor sheet.

According to a second aspect, in the presence determination device according to the first aspect, the absence determination time may be set within a range of 0.5 seconds to 60 seconds.

According to the presence determination device of the aspect, the absence determination time is set within the range, and thereby the determination can be executed with high precision. That is, when the absence determination time is too short, there is concern that an erroneous determination will be executed when false detection results in temporary lowering of a level of the ballistocardiographic signal. In addition, when the absence determination time is too long, there is concern that the absence determination will not be executed even when the user returns back after leaving the detection region once.

According to a third aspect, in the presence determination device according to the first or second aspect, the absence determination unit may execute the absence determination based on comparison between the absence threshold value and a representative value of the ballistocardiographic signal within a predetermined time.

According to the presence determination device of the aspect, the representative value is employed depending on a purpose, and thereby a highly precise determination of presence and absence, a reduction in erroneous determination due to noise, simplification in computation process of presence-absence determination, or the like is achieved.

According to a fourth aspect, in the presence determination device according to the first or second aspect, the absence determination unit may execute the absence determination based on comparison between the absence threshold value and any one of a maximum value, a minimum value, a mean value, a median value, and a sum of the ballistocardiographic signal within a predetermined time.

According to the presence determination device of the aspect, for example, the maximum value of the ballistocardiographic signal within the predetermined time is employed as a value which is compared to the absence threshold value, and thereby a maximum amplitude of a heartbeat waveform can be easily obtained to determine presence and absence with high precision. In addition, when the minimum value, the mean value, or the median value of the ballistocardiographic signal within the predetermined time is employed, an erroneous determination indicating that presence is erroneously determined due to noise during absence is difficult to execute. Alternatively, when the sum of the ballistocardiographic signal is employed, the computation process of the absence determination is simplified, and thereby a processing load is reduced.

According to a fifth aspect, the presence determination device according to any one of the first to fourth aspects may further include a presence determination unit that executes a presence determination indicating that the living body is present on the detection region, based on the ballistocardiographic signal. When a state where the ballistocardiographic signal is higher than a presence threshold value lasts beyond a presence determination time, the presence determination unit may execute the presence determination.

According to the presence determination device of the aspect, even though a level of vibration temporarily increases due to vibration noise or the like from outside in the presence determination, an erroneous determination of immediate presence is not executed, and thus the determination can be executed with higher precision.

According to a sixth aspect, in the presence determination device according to the fifth aspect, the presence determination time may be set within a range of 0.5 seconds to 30 seconds.

According to the presence determination device of the aspect, the presence determination time is set within the range, and thereby the determination can be executed with high precision. That is, when the presence determination time is too short, there is concern that erroneous presence determination will be executed due to the vibration from the outside. In addition, when the presence determination time is too long, there is concern that the presence determination will not be correctly executed even when the user stays in the detection region only for a short time.

According to a seventh aspect, in the presence determination device according to the fifth or sixth aspect, the presence determination unit may execute the presence determination based on comparison between the presence threshold value and a representative value of the ballistocardiographic signal within a predetermined time.

According to the presence determination device of the aspect, the representative value is employed depending on a purpose, and thereby a highly precise determination of presence and absence, a reduction in erroneous determination due to noise, simplification in computation process of presence-absence determination, or the like is achieved.

According to an eighth aspect, in the presence determination device according to the fifth or sixth aspect, the presence determination unit may execute the presence determination based on comparison between the presence threshold value and any one of a maximum value, a minimum value, a mean value, a median value, and a sum of the ballistocardiographic signal within a predetermined time.

According to the presence determination device of the aspect, for example, the maximum value of the ballistocardiographic signal within the predetermined time is employed as a value which is compared to the presence threshold value, and thereby a maximum amplitude of ballistocardiographic waveform can be easily obtained to determine presence and absence with high precision. In addition, when the minimum value, the mean value, or the median value of the ballistocardiographic signal within the predetermined time is employed, an erroneous determination in which presence is erroneously determined due to noise during absence is difficult to execute. Alternatively, when the sum of the ballistocardiographic signal is employed, the computation process of the presence determination is simplified, and thereby a processing load is reduced.

According to a ninth aspect, in the presence determination device according to any one of the fifth to eighth aspects, the presence threshold value and the absence threshold value may be independently set to different values from each other.

According to the presence determination device of the aspect, the presence and the absence can be determined with higher precision.

According to a tenth aspect, in the presence determination device according to the ninth aspect, the presence threshold value may be equal to or larger than three times the absence threshold value.

According to the presence determination device of the aspect, the presence and the absence can be determined with much higher precision.

According to an eleventh aspect, the presence determination device according to any one of the fifth to tenth aspects may further include a body movement determination unit that executes a body movement determination of determining a body movement of the living body on the detection region, based on the ballistocardiographic signal. The body movement determination unit may use a body movement threshold value in the body movement determination, the body movement threshold value being set to a value larger than the presence threshold value. When a value of the ballistocardiographic signal is higher than the body movement threshold value, and the presence determination unit detects the presence of the living body and executes the presence determination for a predetermined time before and after the value of the ballistocardiographic signal is higher than the body movement threshold value, the body movement determination unit may detect the body movement of the living body and executes the body movement determination.

According to the presence determination device of the aspect, the body movement such as turning over in a recumbent position or re-sitting in a sitting position can be detected with higher precision.

According to a twelfth aspect, the presence determination device according to any one of the first to eleventh aspects may further include a signal amplification unit that amplifies the ballistocardiographic signal.

According to the presence determination device of the aspect, the presence determination and the absence determination are executed based on the ballistocardiographic signal amplified by the signal amplification unit, and thereby determination precision is improved.

According to a thirteenth aspect, the presence determination device according to the twelfth aspect may further include a heartbeat waveform computation unit that calculates a heartbeat waveform from the ballistocardiographic signal amplified by the signal amplification unit.

According to the presence determination device of the aspect, when the absence determination is executed based on the ballistocardiographic signal, an effect of the noise or the like can be more suppressed to improve the determination precision by executing the determination using the heartbeat waveform obtained from the ballistocardiographic signal, compared to a case where the determination is executed using the ballistocardiographic signal directly, for example. In addition, the heartbeat waveform can be displayed on a monitor or the like, and thus presence and absence of a user may be determined depending on whether or not the heartbeat waveform is displayed on the monitor.

According to a fourteenth aspect, the presence determination device according to any one of the first to thirteenth aspects may further include a vital frequency analysis unit that performs frequency analysis on the detection signal of the piezoelectric sensor sheet and calculates vital spectra including a power spectrum obtained from ballistocardioaction of the living body. The ballistocardiographic signal acquisition unit may acquire the ballistocardiographic signal based on the vital spectra.

According to the presence determination device of the aspect, even when an amplitude of vibration input to the piezoelectric sensor sheet is low and the detection signal is weak, the presence determination can be executed with high precision based on a frequency analysis result of the detection signal. Consequently, even when the piezoelectric sensor sheet is laid on the lower side of a mattress or the like, and an amplitude of an input to the piezoelectric sensor sheet is low, for example, the presence and absence of the user can be determined with high precision.

According to a fifteenth aspect, in the presence determination device according to the fourteenth aspect, the vital spectra may be power spectra including both a ballistocardiographic spectrum which is the power spectrum obtained from the ballistocardioaction of the living body and a respiration spectrum which is a power spectrum obtained from respiration of the living body.

According to the presence determination device of the aspect, the vital spectra include the respiration spectrum which is obtained to be larger than the ballistocardiographic spectrum, and thereby a large vital spectrum can be obtained. Consequently, even when the detection signal is weak, a strong ballistocardiographic signal which is acquired based on the vital spectra can be obtained such that the presence-absence determination can be executed with high precision.

According to a sixteenth aspect, in the presence determination device according to the fourteenth or fifteenth aspect, the ballistocardiographic signal may be acquired based on a representative value of the vital spectra.

According to the presence determination device of the aspect, the representative value is employed depending on a purpose, and thereby a highly precise determination of presence and absence, a reduction in erroneous determination due to noise, simplification in computation process of presence-absence determination, or the like is achieved.

According to a seventeenth aspect, in the presence determination device according to the sixteenth aspect, the representative value of the vital spectra may be set to any one of a maximum value, a minimum value, a mean value, a median value, and a sum of the vital spectra.

According to the presence determination device of the aspect, for example, the ballistocardiographic signal based on the maximum value of the vital spectra is employed as a value which is compared to the absence threshold value, and thereby the presence and absence can be determined with high precision. In addition, when the ballistocardiographic signal is employed based on the minimum value, the mean value, or the median value of the vital spectra, an erroneous determination in which presence is erroneously determined due to noise during absence is difficult to execute. Alternatively, when the ballistocardiographic signal based on the sum of the vital spectra is employed, the computation process of the absence determination is simplified, and thereby a processing load is reduced.

According to an eighteenth aspect, the presence determination device according to any one of the fourteenth to seventeenth aspects may further include a noise frequency analysis unit that performs frequency analysis on the detection signal of the piezoelectric sensor sheet and calculates a noise spectrum which is a power spectrum in a frequency range higher than a vital frequency range which is a frequency range of the vital spectra. The absence determination unit may execute the absence determination based on comparison between the absence threshold value and an S/N ratio which is a ratio of the ballistocardiographic signal to a noise signal based on the noise spectrum.

According to the presence determination device of the aspect, the presence-absence determination is executed based on the comparison between the absence threshold value and the S/N ratio which is the ratio of the ballistocardiographic signal to the noise signal, and thereby the power spectrum of the vital frequency range is raised due to white noise such that an erroneous determination in which presence is erroneously determined during absence is difficult to execute.

Incidentally, the noise signal is acquired based on a representative value of the noise spectrum. For example, the representative value of the noise spectrum is appropriately selected from a maximum value, a minimum value, a mean value, a median value, a sum, or the like of the noise spectrum. The representative value is employed depending on a purpose, and thereby a highly precise determination of presence and absence, a reduction in erroneous determination due to noise, simplification in computation process of presence-absence determination, or the like is achieved.

According to a nineteenth aspect, in the presence determination device according to any one of the fourteenth to eighteenth aspects, the piezoelectric sensor sheet may be disposed on a lower side of a cushion body on which the living body lies.

According to the presence determination device of the aspect, the cushion body is present on the piezoelectric sensor sheet, and thereby a shift or deformation of the piezoelectric sensor sheet due to the body movement of the user is inhibited. In addition, since a top surface of the piezoelectric sensor sheet is covered with the cushion body, the detection signal is unlikely to receive an effect of wind or the like, even when a highly sensitive piezoelectric sensor sheet is employed.

According to the presence determination device of the present disclosure, an absence determination time is set, and thereby an absence determination of whether or not a living body is present on a detection region can be executed with high precision.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A presence determination device that determines whether or not a living body is present on a detection region, the presence determination device comprising:
a flexible piezoelectric sensor sheet that is laid out in the detection region and outputs a detection signal corresponding to an input vibration;
a ballistocardiographic signal acquisition unit that extracts a ballistocardiographic signal corresponding to a ballistocardioaction from the detection signal of the piezoelectric sensor sheet; and
an absence determination unit that executes an absence determination based on the ballistocardiographic signal, where the absence determination indicates that the living body is not present on the detection region,
wherein, when a state where the ballistocardiographic signal is lower than an absence threshold value lasts beyond an absence determination time, the absence determination unit executes the absence determination.

2. The presence determination device according to claim 1,
wherein the absence determination time is set within a range of 0.5 seconds to 60 seconds.

3. The presence determination device according to claim 1,
wherein the absence determination unit executes the absence determination based on a comparison between the absence threshold value and a representative value of the ballistocardiographic signal within a predetermined time.

4. The presence determination device according to claim 1,
wherein the absence determination unit executes the absence determination based on a comparison between the absence threshold value and any one of a maximum value, a minimum value, a mean value, a median value, and a sum of the ballistocardiographic signal within a predetermined time.

5. The presence determination device according to claim 1, further comprising:
a presence determination unit that executes a presence determination based on the ballistocardiographic signal, in which the presence determination indicates that the living body is present on the detection region,
wherein, when a state where the ballistocardiographic signal is higher than a presence threshold value lasts beyond a presence determination time, the presence determination unit executes the presence determination.

6. The presence determination device according to claim 5,
wherein the presence determination time is set within a range of 0.5 seconds to 30 seconds.

7. The presence determination device according to claim 5,
wherein the presence determination unit executes the presence determination based on a comparison between the presence threshold value and a representative value of the ballistocardiographic signal within a predetermined time.

8. The presence determination device according to claim 5,
wherein the presence determination unit executes the presence determination based on a comparison between the presence threshold value and any one of a maximum value, a minimum value, a mean value, a median value, and a sum of the ballistocardiographic signal within a predetermined time.

9. The presence determination device according to claim 5,
wherein the presence threshold value and the absence threshold value are independently set to different values from each other.

10. The presence determination device according to claim 9,
wherein the presence threshold value is equal to or larger than three times the absence threshold value.

11. The presence determination device according to claim 5, further comprising:
a body movement determination unit that executes a body movement determination of determining a body movement of the living body on the detection region based on the ballistocardiographic signal,
wherein the body movement determination unit uses a body movement threshold value in the body movement determination, the body movement threshold value being set to a value larger than the presence threshold value, and
wherein, when a value of the ballistocardiographic signal is higher than the body movement threshold value, and the presence determination unit detects presence of the living body and executes the presence determination for a predetermined time before and after the value of the ballistocardiographic signal is higher than the body movement threshold value, the body movement determination unit detects the body movement of the living body and executes the body movement determination.

12. The presence determination device according to claim 1, further comprising:
a signal amplification unit that amplifies the ballistocardiographic signal.

13. The presence determination device according to claim 12, further comprising:
a heartbeat waveform computation unit that calculates a heartbeat waveform from the ballistocardiographic signal amplified by the signal amplification unit.

14. The presence determination device according to claim 1, further comprising:
a vital frequency analysis unit that performs a frequency analysis on the detection signal of the piezoelectric sensor sheet and calculates vital spectra including a power spectrum obtained from ballistocardioaction of the living body,
wherein the ballistocardiographic signal acquisition unit acquires the ballistocardiographic signal based on the vital spectra.

15. The presence determination device according to claim 14,
wherein the vital spectra are power spectra including both a ballistocardiographic spectrum which is the power spectrum obtained from the ballistocardioaction of the living body and a respiration spectrum which is a power spectrum obtained from respiration of the living body.

16. The presence determination device according to claim 14,
wherein the ballistocardiographic signal is acquired based on a representative value of the vital spectra.

17. The presence determination device according to claim 16,
wherein the representative value of the vital spectra is set to any one of a maximum value, a minimum value, a mean value, a median value, and a sum of the vital spectra.

18. The presence determination device according to claim 14, further comprising:
a noise frequency analysis unit that performs a frequency analysis on the detection signal of the piezoelectric sensor sheet and calculates a noise spectrum which is a power spectrum in a frequency range higher than a vital frequency range which is a frequency range of the vital spectra,
wherein the absence determination unit executes the absence determination based on a comparison between the absence threshold value and an S/N ratio which is a ratio of the ballistocardiographic signal to a noise signal based on the noise spectrum.

19. The presence determination device according to claim 14,
wherein the piezoelectric sensor sheet is disposed on a lower side of a cushion body on which the living body lies.
